(19) United States
(12) Patent Application Publication
(10) Pub. No.: US 2023/0022098 A1
(43) Pub. Date: Jan. 26, 2023

(54) SAMPLE-CONCENTRATING ASSISTED ARRAY-BASED ASSAY METHOD

(71) Applicant: SENTILUS HOLDCO, LLC, Norcross, GA (US)

(72) Inventors: Hui HUANG, Norcross, GA (US); Jigar PATEL, Norcross, GA (US); Kejalben GHIWALA, Norcross, GA (US); Jiacheng YANG, Norcross, GA (US)

(21) Appl. No.: 17/624,485

(22) PCT Filed: Jul. 2, 2020
(86) PCT No.: PCT/US2020/040688

§ 371 Date: Jan. 3, 2022

Related U.S. Application Data
(60) Provisional application No. 62/870,205, filed on Jul. 3, 2019.

(51) Int. Cl. G01N 33/552; G01N 33/543

(52) U.S. Cl. CPC G01N 33/552; G01N 33/54386; G01N 2470/04

(57) ABSTRACT

The present invention relates to methods for detecting a target molecule in a liquid sample. The methods described herein comprise applying at least a portion of the liquid sample on a solid substrate that comprises a non-fouling polymer layer, decreasing the atmospheric pressure surrounding the solid substrate containing the portion of the liquid sample for a time sufficient for a majority of the liquid to evaporate from the portion of the liquid sample applied to the solid substrate, contacting the liquid sample with one or more binding agents that binds to the target molecule after the majority of the liquid has evaporated from the liquid sample, and detecting the presence of the one or more binding agents on the solid substrate, wherein the presence of the one or more binding agents indicates the presence of the target molecule in the liquid sample.

### STATEMENT REGARDING FEDERALLY SPONSORED RESEARCH OR DEVELOPMENT

### BACKGROUND OF THE INVENTION

Array-based biological assay is typically carried out in liquid phase where sample containing the targets to be detected is brought to the substrate with pre-spotted array of capture reagents (probes). During sample incubation on the array substrate, the target components from the sample diffuse towards the array surface and be captured. The diffusion process is both time consuming and inefficient. Consequently, some assays take long time to finish. In addition, due to the low efficiency, only a small fraction of the targets from the sample solution can be captured on the array surface. Therefore, it may require large sample volume for some assays, especially when low target concentration needs to be detected.

Sample evaporation is another severe problem for liquid phase assay. This could add significant difficulty if long incubation time is required or multiple samples need to be tested in a batch. In a batch format, for example, 96-well plate format, when multiple samples are sequentially added to the wells, there is significant time lapse between the first and the last sample added to the plate. During the time lapse, the samples loaded earlier could evaporate and result higher concentration than the samples added later. This could cause considerable nonuniformity of the result across the plate.

The sample-concentrating assisted array-based assay method (SCA) provided in the present invention can largely resolve the problems stated above for liquid phase assay.

### BRIEF SUMMARY OF THE INVENTION

The present invention relates to methods for detecting a target molecule in a liquid sample. The methods described herein comprise applying at least a portion of the liquid sample on a solid substrate that comprises a non-fouling polymer layer, and decreasing the atmospheric pressure surrounding the solid substrate containing the portion of the liquid sample for a time sufficient for a majority of the liquid to evaporate from the portion of the liquid sample applied to the solid substrate. Moreover, the methods comprise contacting the liquid sample with one or more binding agents that bind to the target molecule after the majority of the liquid has evaporated from the liquid sample, and detecting the presence of the one or more binding agents on the solid substrate, wherein the presence of the one or more binding agents indicates the presence of the target molecule in the liquid sample.

### BRIEF DESCRIPTION OF THE DRAWINGS/FIGURES

FIG. 1 is a diagram of an exemplary process of the sample-concentrating assisted array-based assay method.

FIG. 2 depicts a Ferritin quantitative sandwich assay using the methods of the invention. Antibody specific to human-Ferritin (capture Ab) is pre-spotted as array on epoxy-copoly(oligo(ethylene glycol) methyl ether methacrylate (e-POEGMA) coated substrate. Step-1: Human plasma/serum sample is placed on the array for the Ferritin content to be captured by the capture Ab; Step-2: the fluorophore-labeled detection antibody is added to label the captured Ferritin. The remaining components are washed away to result the clean array surface for fluorescent quantification.

FIG. 3 depicts the silicone adaptors for array-based assay (Grace Bio-Labs, Bend, Oregon).

FIG. 4 depicts the assay intensity with recombinant Ferritin at 0, 6, 13, 25, 50, 100, 200, and 400 ng/mL. The bar chart on left is for a wet assay using 7 µL sample solutions; the one on right is for assay with SCA method (dry assay), using 4 µL sample solutions. The 3 bars from left to right in each group indicate the detection antibody concentration of 3, 6, and 9 microgram/mL, respectively. Error bar indicates 95% confidence interval of the intensity.

FIG. 5 depicts the scatter plot of Ferritin assay intensity from FIG. 4. The fit curves are 2^{nd} order polynomial. Error bar indicates 95% confidence interval of the intensity.

FIG. 6 depicts a diagram of the double-antigen bridging immunoassay.

FIG. 7 depicts Treponema pallidum (Tp) and CMV antibodies detection with double-antigen bridging immunoassay. Error bar indicates 95% confidence interval of the intensity.

FIG. 8A depicts the comparison of intensities from Ferritin assays with and without storage (3 days). Scatter plot of sample intensities for the slide stored over 3 days (y-axis) and that without storage (x-axis). Each point is from the same sample. Samples are Ferritin plasma/serum 1:2 diluted with TSB. Error bar indicates 95% confidence interval of the intensity. FIG. 8B depicts the comparison of intensities from Ferritin assays with and without storage (3 days). Scatter plot of sample intensities for the slide stored over 3 days (y-axis) and that without storage (x-axis). Each point is from the same sample. Samples are recombinant Ferritin standard from world health organization (WHO) diluted with TSB. Error bar indicates 95% confidence interval of the intensity.

FIG. 9 depicts an exemplary IL-6 titration curve. Error bar is 95% confidence interval.

FIG. 10 depicts the average fluorescence assay intensity for A-antigen and B-antigen microarray spots (Error bar is 95% confidence interval).

### DETAILED DESCRIPTION OF THE INVENTION

The present invention relates to methods for detecting a target molecule in a liquid sample. The methods described herein comprise applying at least a portion of the liquid sample on a solid substrate that comprises a non-fouling polymer layer, and decreasing the atmospheric pressure surrounding the solid substrate containing the portion of the liquid sample for a time sufficient for a majority of the liquid to evaporate from the portion of the liquid sample applied to the solid substrate. Moreover, the methods comprise contacting the liquid sample with one or more binding agents that binds to the target molecule after the majority of the liquid has evaporated from the liquid sample, and detecting the presence of the one or more binding agents on the solid substrate, wherein the presence of the one or more binding agents indicates the presence of the target molecule in the liquid sample.

As used herein, a target molecule means any molecule or compound of interest. A target molecule can be, but is not limited to, a protein, peptide, carbohydrate, polysaccharide, glycoprotein, hormone, receptor, antigen, antibody, virus, substrate, metabolite, transition state analog, cofactor, inhibitor, drug, dye, nutrient, growth factor, etc., without limitation. In some embodiment, the target molecule is a cell, a small molecule ligand, a lipid, a carbohydrate, a polynucleotide, a peptide, a protein, an antigen, an antibody, or a combination thereof.

As used herein, the term antigen refers to any substance or specific portions thereof that specifically binds to an antibody, T-cell receptor or other component of the immune system either alone or after forming a complex with a larger molecule, such as a protein. In some embodiments, the antigen is a peptide or portion thereof. In other embodiments, the antigen is a carbohydrate, such as sugar. In some embodiments, the antigen is foreign to the body. In other embodiments, the antigen is a natural product of the body. The antigens used in the present invention need not be the entire molecule. For example, an antigen can be a short amino acid sequence of a larger protein molecule, and this short sequence is responsible for the binding of the protein to the immune system component.

In some embodiments, the target molecule is a blood type antigen, a platelet antigen, an infectious disease antigen, a human leukocyte antigen (HLA), an interleukin antigen, or any combination thereof. In specific embodiments, the target molecule is a human immune deficiency virus (HIV) antigen, a hepatitis B virus (HBV) antigen, a hepatitis C virus (HCV) antigen, a human T-lymphotropic virus (HTLV) antigen, a Treponema pallidum (TP) antigen, or any combination thereof.

In some embodiments, the target molecule is one or more of a human A blood type antigen, a human B blood type antigen, a human Rh factor antigen, a human MNS blood type antigen, a human P blood type antigen, a human P1PK blood type antigen, a human Lutheran blood type antigen, a human Kell blood type antigen, a human Lewis blood type antigen, a human Duffy blood type antigen, a human Kidd blood type antigen, a human Diego blood type antigen, a human Yt or Cartwright blood type antigen, a human Xg blood type antigen, a human Scianna blood type antigen, a human Dombrock blood type antigen, a human Colton blood type antigen, a human Landsteiner-Wiener blood type antigen, a human Chido/Rodgers blood type antigen, a human H blood type antigen, a human Hh/Bombay blood type antigen, a human Kx blood type antigen, a human Gerbich blood type antigen, a human Cromer blood type antigen, a human Knops blood type antigen, a human Indian blood type antigen, a human Ok blood type antigen, a human Raph blood type antigen, a human John Milton Hagen blood type antigen, a human I blood type antigen, a human li blood type antigen, a human Globoside blood type antigen, a human Gill blood type antigen, a human Rh-associated glycoprotein blood type antigen, a human Forssman blood type antigen, a human Langereis blood type antigen, a human Junior blood type antigen, or any combination thereof.

The target molecules, if present, will be in the liquid sample. As used herein, a liquid sample refers to any sample in the liquid or fluid form. In some embodiments, the liquid sample is a biological sample, meaning that the sample is of biological origin, i.e., obtained from a biological specimen, such as but not limited to an animal. In some embodiments, the liquid sample is a sample of blood, serum, plasma, lymph fluid, bile fluid, urine, saliva, mucus, sputum, tears, cerebrospinal fluid (CSF), bronchioalveolar lavage, nasopharyngeal lavage, rectal lavage, vaginal lavage, colonic lavage, nasal lavage, throat lavage, synovial fluid, semen, ascites fluid, pus, maternal milk, ear fluid, sweat, or amniotic fluid obtained from a mammal, such as but not limited to a human or non-human primate. Further, the sample may be mixed with one or more chemicals, reagents, diluents, or buffers.

In other embodiments, the sample may be processed prior to performing the methods of the present invention on the sample. For example, a sample may be obtained from an animal, e.g., a blood sample, and the sample may be diluted, concentrated, filtered, centrifuged, frozen and thawed, mixed with another component, etc. Regardless of the processing prior to application of the methods of the invention described herein, the processed sample would still be considered “the sample” for the purposed of the present invention.

The sample that is taken directly from the biological source need not be liquid. For example, a tissue extract can be taken from a subject and this solid sample can be processed, e.g., ground or minced, etc., in the presence of liquid nitrogen, buffers, enzymes, etc. to produce a liquid sample. This liquid sample can then be further processed according to some or any of the processing methods described herein above.

In some embodiments, the entirety of a liquid sample is applied on a solid substrate. In other embodiments, only a portion of the liquid sample is applied on a solid substrate.

The methods of the present invention also contemplate embodiments in which the sample is “collected” directly onto the solid substrate. In other words, the methods of the present invention encompass select embodiments in which there is no sample collection step prior to applying the sample onto the solid substrate. In other embodiments, a sample is collected in a separate container prior to applying at least a portion onto the solid substrate. If collected into a separate container, the sample may be stored for a period of time prior to applying at least a portion of the sample to solid substrate. For example, the sample may be collected in a laboratory or a hospital. The sample may be collected by a technician, a nurse, or a physician. The sample may be collected in any form, such as liquid or solid, and stored by a means deemed appropriate by a skilled person. In some embodiments, a portion of the sample, e.g., a liquid sample, can be directly applied to the solid substrate. In other embodiments, the sample can be reconstituted into a liquid sample and a portion of the reconstituted liquid sample can be applied to the solid substrate.

As used herein, a solid substrate encompasses a variety of different types of substrates. In some embodiments, the solid substrate may be organic or inorganic, it may be metal (e.g., copper or silver) or non-metal, or it may be a polymer or nonpolymer. In other embodiments, the solid substrate may be conducting, semiconducting or nonconducting (insulating), it may be reflecting or nonreflecting, or it may be porous or nonporous. In other embodiments, the solid substrate may comprise polyethylene, polytetrafluoroethylene, polystyrene, polyethylene terephthalate, polycarbonate, gold, silicon, silicon oxide, silicon oxynitride, indium, tantalum oxide, niobium oxide, titanium, titanium oxide, platinum, iridium, indium tin oxide, diamond or diamond-like film, etc. In other embodiments, the solid substrate may be a substrate suitable for “chip-based” and “pin-based” combinatorial chemistry techniques. In one specific example, the solid substrate on which the liquid sample is applied is an array, such as a microarray. In another specific example, the solid substrate on which the liquid sample is applied may be a chip. The solid substrate as used in the present invention can be prepared in accordance with known techniques.

In some embodiments, the solid substrate includes but is not limited to metals, metal oxides, alloys, semiconductors, polymers (such as organic polymers in any suitable form including woven, nonwoven, molded, extruded, cast, etc.), silicon, silicon oxide, a plastic, ceramics, glass, and composites thereof.

The methods described herein comprise decreasing the atmospheric pressure surrounding the solid substrate containing at least a portion of the liquid sample for a time sufficient for a majority of the liquid to evaporate from the portion of the liquid sample applied to the solid substrate. As used herein, atmospheric pressure means the pressure exerted by the environment and immediately surrounding the solid substrate on which the liquid sample has been applied. For example, the atmosphere pressure may be the air pressure within a closed container, such as, but not limited to a cold storage room, a refrigerator, a desiccator, a cell culture incubator, etc. The air pressure immediately surrounding the substrate and sample portion need not be the same as the actual atmospheric pressure of the surrounding climate. In some embodiments, the solid substrate containing a portion of the liquid sample is transferred into a sealable enclosure prior to decreasing the surrounding atmospheric pressure. For example, the solid substrate containing the portion of the liquid sample is transferred to a desiccator prior to lowering the atmospheric pressure surrounding the solid substrate. In some embodiments, the sealable enclosure is connected with a vacuum or a device that can lower the atmospheric pressure surrounding the solid substrate.

In specific embodiments, decreasing the atmospheric pressure surrounding the solid substrate can be performed under a controlled temperature or a specific temperature range. In some embodiments, the temperature is lower than or equal to about 4° C. when the atmospheric pressure is decreased. In other embodiments, the temperature is from about 4° C. to about 40° C. when the atmospheric pressure is decreased. In more specific embodiments, the temperature is from about 20° C. to about 25° C. when the atmospheric pressure is decreased.

In some embodiments, the atmospheric pressure surrounding the solid substrate containing a portion of the liquid sample is reduced to from about 0 to about 2000 millibar. In other embodiments, the atmospheric pressure surrounding the solid substrate containing a portion of the liquid sample is reduced to from about 0 to about 1000 millibar. In more specific embodiments, the atmospheric pressure surrounding the solid substrate containing a portion of the liquid sample is reduced to from about 0 to about 500 millibar. In still more specific embodiments, the atmospheric pressure surrounding the solid substrate containing a portion of the liquid sample is reduced to from about 0 to about 200 millibar.

The methods herein comprise decreasing the atmospheric pressure surrounding the solid substrate containing the portion of the liquid sample for a time sufficient for a majority of the liquid to evaporate from the portion of the liquid sample applied to the solid substrate. As used herein, a sufficient time refers to a period of time that is long enough for a majority of the liquid to evaporate from the portion of the liquid sample applied to the solid substrate, upon visual inspection. For example, the liquid sample can be dried to a state when no residual liquid can be detected visually (i.e. with the naked eye or via microscope). In some embodiments, the pressure is lowered for a time that is longer than when the liquid is no longer present upon visual inspection. In some embodiments, the time sufficient for a majority of the liquid to evaporate from the portion of the liquid sample applied to the substrate is about 10 minutes. In other embodiments, the time sufficient for a majority of the liquid to evaporate from the portion of the liquid sample applied to the substrate is less than about 10 minutes. In specific embodiments, the time sufficient for a majority of the liquid to evaporate from the portion of the liquid sample applied to the substrate is about 5 minutes or less. In more specific embodiments, the time sufficient for a majority of the liquid to evaporate from the portion of the liquid sample applied to the substrate is about 2 minutes or less. In one specific embodiment, the time sufficient for a majority of the liquid to evaporate from the portion of the liquid sample applied to the substrate is about 1 minute. In another specific embodiment, the time sufficient for a majority of the liquid to evaporate from the portion of the liquid sample applied to the substrate is about 1 to about 2 minutes. However, times longer than about 10 minutes are also contemplated by the present invention.

The pressure is decreased until a majority of the liquid has evaporated. As used herein, a “majority” means at least 50% of the liquid is evaporated from the portion of the liquid sample on the solid substrate. In specific embodiments, more than 50% of the liquid is evaporated from the portion of the liquid sample on the solid substrate. In more specific embodiments, more than 75% of the liquid is evaporated from the portion of the liquid sample on the solid substrate. In even more specific embodiments, more than 90% of the liquid is evaporated from the portion of the liquid sample on the solid substrate. In still mores specific embodiments, more than 99% of the liquid is evaporated from the portion of the liquid sample on the solid substrate. The moisture content of the solid substrate and sample after decreasing the pressure need not be quantified or assessed, beyond a visual inspection.

The methods of the present invention do not require a wash or rinse step after application of the sample onto the substrate. In some embodiments, the methods encompass applying and drying the sample on the solid substrate and adding a binding agent directly onto the sample that is dried on the substrate.

In some embodiments, the solid substrate used herein comprises a surface that allows for the application of a polymer layer. In some embodiments, the polymer layer as described herein exhibit non-fouling properties. The terms “non-fouling” and “antifouling” are used interchangeably herein. The term “non-fouling,” as used herein with respect to the polymer layer, is used as it is in the art and generally means surfaces that resist adsorption of proteins and/or adhesion of cells. The non-fouling property of the polymer can be introduced by any suitable method such as, for example, incorporation of a non-fouling agent or by the structure/architecture of the polymer itself. Non-fouling agents are known in the field and one of skill can select a specific agent depending on the particular use of device, or on the availability of the non-fouling agent. Non-limiting examples of non-fouling agents include but are not limited to organic and inorganic compounds having biocidal activity, as well as compounds that can be incorporated with or bound to the polymer layer that reduce or inhibit non-specific binding interaction of a biomolecule (e.g., cell, protein, nucleotide, carbohydrate, or lipid) with the polymer upon contact.

In specific embodiments, a polymer layer has a structure or architecture that provides a non-fouling property. In some embodiments, the polymer may suitably include brush polymers, which are generally formed by the polymerization of monomeric core groups having one or more groups that function to inhibit binding of a biomolecule (e.g., cell, protein, nucleotide, carbohydrate, or lipid) coupled thereto. Suitably, the monomeric core group can be coupled to a protein-resistant head group. In some embodiments, the non-fouling polymer layer of the methods provided herein comprises a brush polymer comprising a polymeric stem and a multitude of molecular bristles projecting from said polymeric stem, wherein the brush polymer comprises a co-polymer of an oligo ethylene glycol methacrylate (OEGMA) monomer and a methacrylate monomer (MAM) comprising a linking moiety and an electrophilic head group. In some embodiments, the co-polymer comprises a MAM to OEGMA v/v ratio from about 1:3 to about 1:8. In certain specific embodiments, the MAM to OEGMA v/v ratio is about 1:4. In some embodiments, the OEGMA comprises poly(ethylene glycol) methacrylate (PEGMA) and poly(ethylene glycol) methyl ether methacrylate (PEGMEM). In some embodiments, the electrophilic head group is an epoxide group or an epoxy-ketone group. In some embodiments, the MAM is glycidyl methacrylate (GMA). In some embodiments, the non-fouling polymer layer comprises a co-polymer of epoxy-co-poly(oligo(ethylene glycol) methyl ether methacrylate (epoxy-co-POEGMA, or e-POEGMA). In some embodiments, the co-polymer comprises GMA and PEGMEM, and wherein the GMA to PEGMEM v/v ratio is about 1:4.

In some of the embodiments described herein, the polymer layer can be formed by surface-initiated ATRP (SI-ATRP) of oligo(ethylene glycol)methyl methacrylate (OEGMA) to form a poly(OEGMA) (POEGMA) film. In an embodiment, the polymer layer is a functionalized POEGMA film prepared by copolymerization of a methacrylate and methoxy terminated OEGMA.

In general, the brush molecules can be from 2 or 5 up to 100 or 200 nanometers in length, or more, and can be deposited on the surface portion at a density of from 10, 20 or 40 to up to 100, 200 or 500 milligrams per meter, or more. Protein resistant groups can be hydrophilic head groups or kosmotropes. Examples can include but are not limited to oligosaccharides, tri(propyl sulfoxide), hydroxyl, glycerol, phosphorylcholine, tri(sarcosine) (Sarc), N-acetylpiperazine, betaine, carboxybetaine, sulfobetaine, permethylated sorbitol, hexamethylphosphoramide, an intramolecular zwitterion (for example,— CH₂N⁺(CH₃)₂CH₂CiI₂CH₂SO₃") (ZW), and mannitol.

Additional examples of kosmotrope protein resistant head groups can include, but are not limited to:

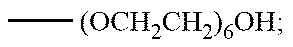

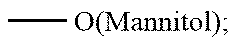

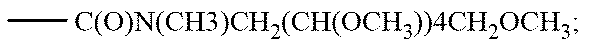

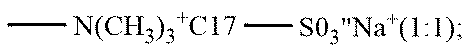

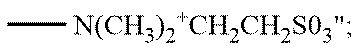

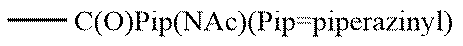

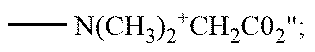

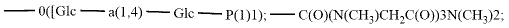

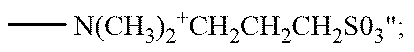

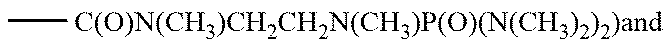

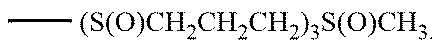

In some of the embodiments described herein, a suitable protein resistant head group can comprise poly(ethylene glycol) (PEG), for example, PEG of from 3 to 20 monomelic units.

In certain embodiments, the e-POEGMA used in the methods described herein is synthesized by adopting procedures known for the synthesis of non-functionalized POEGMA. In certain embodiments, the POEGMA coating technology is modified by adding reactive functional groups (e.g., epoxide groups) to the POEGMA composition during the polymerization process. The functional groups provide covalent chemical bonding for the immobilization of biomolecules without compromising the non-fouling characteristics of the POEGMA surface. In certain embodiments, the e-POEGMA is synthesized from a mixture of PEGMEM and glycidyl methacrylate (GMA, MW 142). In the copolymer, PEGMEM contributes non-fouling properties while GMA contributes chemical bonding capabilities. Since GMA is not soluble in pure water, certain amount of ethanol is added to form homogeneous mixture. Typically, about 10-20% ethanol is sufficient, depending on the concentrations of monomers in the polymerization mixture.

In some embodiments, the solid substrate comprises a plurality of capture regions. As used herein, the term capture region refers to the area or regions on the solid substrate surface where one or more capture agents are bound. The non-fouling polymer layer may only be on the solid substrate where the capture regions are located or the non-fouling polymer layer may be applied to the entire surface. The number of capture regions can vary widely and can depend on several factors including the size and shape of the solid substrate, the intended use of the method (e.g., a point-of-care diagnostic, a panel array (e.g., microarrays for screening DNA, MM Chips (microRNAs), protein, tissue, cellular, chemical compounds, antibody, carbohydrate, etc.), and the like. The methods of the present invention described herein are not dependent on the number and arrangement of the capture regions, if present.

In some embodiments, each of the plurality of capture regions comprises one or more populations of capture agents, wherein each distinct population of capture agents can specifically bind to a specific target molecule. In some embodiments, each of the plurality of capture region comprises at least one distinct population of capture agents. In other embodiments, each of the plurality of capture region comprises more than one distinct population of capture agents. The capture agents can be covalently or non-covalently bound to the capture regions of the polymer layer.

As used herein, the term capture agent refers to any agent that can specifically bind to the target molecule in the liquid sample. Examples of suitable capture agents can include, but are not limited to, cells, small molecule ligands, lipids, carbohydrates, polynucleotides, peptides, proteins, antigens, antibodies, antibody fragments and the like, or a combination thereof.

By “specifically binds,” it generally means that the capture agent binds to the target molecule with higher affinity than it binds to other molecules. For example, a capture agent is said to “specifically bind” to a target molecule when it binds to that target molecule, via its specific binding domain, more readily than it would bind to a random, unrelated molecule.

In some embodiments, the capture agent can comprise a biomarker associated with any disease, disorder, or biological state of interest. Accordingly, the selection of the capture agent can be driven by the intended use or application of the methods described herein and can include any molecule known to be associated with a disease, disorder, or biological state of interest, or any molecule suspected of being associated with a disease, disorder, or biological state of interest. Thus, the selection of a capture agent is within the ability of one skilled in the art, based on the available knowledge in the art.

In some of the embodiments described herein, the capture agent can comprise a biomarker associated with any microbial infection of interest, examples of which can include but are not limited to: Anthrax, Avian influenza, Botulism, Buffalopox, Chikungunya, Cholera, Coccidioidomycosis, Creutzfeldt- Jakob disease, Crimean-Congo haemorrhagic fever, Dengue fever, Dengue haemorrhagic fever, Diphtheria, Ebola haemorrhagic fever, Ehec ( E. Coli 0157), Encephalitis, Saint-Louis, Enterohaemorrhagic escherischia coli infection Enterovirus, Foodborne disease, Haemorrhagic fever with renal syndrome, Hantavirus pulmonary syndrome, Hepatitis, Human Immunodeficiency Virus (HIV), Influenza, Japanese encephalitis, Lassa fever, Legionellosis, Leishmaniasis, Leptospirosis, Listeriosis, Louseborne typhus, Malaria, Marburg haemorrhagic fever, Measles, Meningococcal disease, Monkeypox, Myocarditis Nipah virus, O’Nyong-Nyong fever, Pertussis, Plague, Poliomyelitis, Rabies, Relapsing fever, Rift Valley fever, Severe acute respiratory syndrome (SARS), Shigellosis, Smallpox vaccine—accidental exposure, Staphylococcal food intoxication, Syphilis, Tularaemia, Typhoid fever, West Nile virus, Yellow fever, etc.

Further, the capture agent can be deposited on the polymer layer by any suitable technique such as microprinting or microstamping, including but not limited to piezoelectric or other forms of non-contact printing and direct contact quill printing. When the capture agent is printed on to the polymer layer, it can suitably be absorbed into the polymer layer such that it remains bound when the solid substrate comprising the polymer layer is exposed to a liquid sample.

As samples are usually dried on the solid substrate after the concentrating step, they are stable for a period of time, which relaxes the time constraint for the assay. When the waiting time stretches to days or months, the methods provided herein practically included sample storage function, where the dried samples on the substrate can be stored for future detection. In certain embodiments, the methods provided herein eliminates additional needs for sample storage. In other embodiments, the methods provided herein can potentially avoid error in transcription. In yet other embodiments, the methods provided herein can aid sample tracking. The advantages provided by the methods disclosed herein can be attributed to less sample handling and transfer steps.

When multiple samples are accessed, there is potential lag between the time when the first and last samples are retrieved and placed on the testing substrate. In a typical wet assay, time difference means difference for incubation time, which could affect the assay signal. In addition, evaporation could affect the actual concentration of the samples thus impact the accuracy of quantitative assay. The SCA methods provided herein solves the problems because it allows flexible time for the first step to load all samples and eliminates the potential assay variation due to finite sample loading time on the substrate. Therefore, the SCA methods provided herein will help batch process with high throughput assays. For example, the SCA methods provided herein can be used for batch-collection of samples before the detection step.

In certain embodiments, the methods provided herein comprise storing the sample on the solid substrate for a period of time after the majority of the liquid has evaporated from the portion of the liquid sample applied to the solid substrate and before contacting the liquid sample with one or more binding agents. In some embodiments, the period of time is about 1 hour, about 2 hours, or up to about 24 hours. In some embodiments, the period of time is about 2 days, 3 days, or up to 10 days. However, the methods provided herein also contemplate that, after the majority of the liquid has evaporated from the portion of the liquid sample applied to the solid substrate and before contacting the liquid sample with one or more binding agents, the sample can be stored on the solid substrate indefinitely. The solid substrate on which the sample has been applied directly and the majority of the liquid evaporated can be stored at room temperature (about 23° C.), or can be stored at higher or cooler temperatures. In select embodiments, the solid substrate on which the sample has been applied directly and the majority of the liquid evaporated is stored at a temperature of between about 40° C. and 35° C., between about 35° C. and 30° C., between about 30° C. and 25° C., between about 25° C. and 20° C., between about 20° C. and 15° C., between about 15° C. and 10° C., between about 10° C. and 5° C., between about 5° C. and 0° C., between about 0° C. and -10° C., between about -10° C. and -20° C., between about -20° C. and -30° C., between about -30° C. and -40° C., between about -40° C. and -50° C., between about -50° C. and -60° C., between about -60° C. and -70° C., or between about -70° C. and -80° C.

In some embodiments, the methods provided herein comprise contacting the liquid sample with one or more binding agents that specifically binds to the target molecule after the target molecule has been captured by the capture agent.

As used herein, a binding agent refers to a molecule that specifically recognizes and binds to a target molecule of interest. Non-limiting examples of binding agents include any molecules that can form immunocomplexes with a target molecule. For example, in one specific embodiment, the target molecule may be an antibody or antibody fragment, and the binding agent would be an antigenic molecule, such as but not limited to a polypeptide, to which the antibody or fragment thereof would bind specifically. In another specific embodiment, the target molecule may be an antigen, and the binding agent in this instance would be an antibody or antigen-binding fragment thereof that specifically binds to the antigen.

After application of the binding agent, the methods of the present invention also contemplate embodiments in which the solution containing binding agent is incubated on the solid substrate containing the dried liquid sample for a period of time. In certain embodiments, the solution containing binding agent is incubated on the solid substrate containing the dried liquid sample for about 10 minutes. In certain embodiments, the solution containing binding agent is incubated on the solid substrate containing the dried liquid sample for less than about 10 minutes. However, incubation times longer than 10 minutes are also contemplated by the present invention. The length of the incubation time can be determined by a person skilled in the art.

In some embodiments, the solution containing binding agent is incubated on the solid substrate containing the dried liquid sample under controlled temperature and a temperature range. In a specific embodiment, the solution containing binding agent is incubated on the solid substrate at 37° C. In another specific embodiment, the solution containing binding agent is incubated on the solid substrate at room temperature, i.e., from about 20° C. to about 25° C. In other embodiments, the solution containing binding agent is incubated on the solid substrate at a temperature lower than 20° C., for example at a temperature of between about 20° C. and about 4° C.

In some embodiments, the solution containing binding agent is incubated on the solid substrate containing the dried liquid sample under static condition. In other embodiments, the solution containing binding agent is incubated on the solid substrate containing the dried liquid sample with gentle shaking, for example, to ensure even distribution of the binding agents over the dried liquid sample on the solid substrate. In specific embodiments, the gentle shaking is achieved by placing the solid substrate on an orbital shaker. However, the methods described herein contemplate any type of instrument that is commonly used in the field, such as but limited to, a vortex shaker, a platform shaker, or an incubator shaker. In certain embodiments, the shaking is set to a fixed speed. In specific embodiments, the shaker is set at lower than 100 rpm, about 100 rpm, about 300 rpm, about 500 rpm, or greater. The speed can be determined by a person skilled in the art and may varied depending on the instrument.

After application of the binding agent, the methods of the present invention also contemplate embodiments in which the solution containing the binding agent is rinsed or washed after the binding agent has been permitted to specifically bind to the target molecule. In some embodiment, the solution containing the binding agent is rinsed or washed to remove unbound binding agent. In some embodiment, the solution containing the binding agent is rinsed or washed to remove excessive binding agent. It can be appreciated by persons skilled in the art that the presence of unbound or excessive binding agents may interfere with the assay. The washing is generally performed by adding a washing buffer to the solid substrate. Washing buffers are commonly used in the art and are available with different options. In some embodiments, the washing buffer comprises one or more of the following ingredients: water, salt, and detergent. In some embodiments, the washing buffer comprises all of the following ingredients: water, salt, and detergent. In other embodiments, the washing buffer may comprise additional ingredients such as chelators, protease inhibitors, bovine serum albumin (BSA), etc. In some embodiments, the washing buffer is TRIS based. For example, in one embodiment, the washing buffer comprises TRIS-buffered saline (TBS). In some embodiments, the washing buffer is phosphate based. In one specific embodiment, the washing buffer comprises phosphate-buffered saline (PBS). In some embodiments, the washing buffer one or more detergents. In other embodiments, the washing buffer does not contain detergent. The detergent used herein can be ionic or nonionic. Examples of commonly used detergents include, but are not limited to, SDS, Triton X-100, CHAPS, NP-40, maltosides, glucosides, Tween-20, Tween-80, and digitonin. The concentration of the detergent can be determined by those skilled in the art. In some embodiments, the washing buffer comprises about 1% of detergent or more. In some embodiments, the washing buffer comprises about 0.5% to about 1% of detergent. In some embodiments, the washing buffer comprises about 0.1% to about 0.5% of detergent. In some embodiments, the washing buffer comprises about 0.01% to about 0.1% of detergent. In other embodiments, the washing buffer comprises about 0.01% of detergent or less. In a specific embodiment, the washing buffer is a PBS containing about 0.01% to about 0.5% tween-20. In a specific embodiment, the washing buffer is a PBS containing about 0.1% tween-20.

In some embodiments, the methods comprise more than one “washing” of the solution containing the binding agent. In a specific embodiment, the methods comprise 3 washings of the solution containing the binding agent. In other embodiments, the methods comprise 2, 3, 4, or more washings of the solution containing the binding agent. In some embodiments, the washing is carried out by hand, for example, using a multi-channel pipettor and vacuum manifold. In other embodiments, the washing is performed by an instrument commonly used in the field. In some embodiments, the washing is conducted by incubation with agitation. In other embodiments, the washing is conducted by incubation without agitation. A person skilled in the art can choose the appropriate washing solution and conditions to achieve the desired outcome.

In some embodiments, the binding agent is labeled with a detectable label that, directly or indirectly, provides a detectable signal. Exemplary detectable labels can include, but are not limited to, chromophores, radiolabels, polynucleotides, small molecules, enzymes, nanoparticles, a quantum dot, or upconverters. In some embodiments, the detectable label can be a fluorophore such as but not limited to a cyanine (e.g., CyDyes such as Cy3 or Cy5), a fluorescein, a rhodamine, a coumarin, a fluorescent protein or functional fragment thereof. In some embodiments, the detectable label can comprise gold, silver, or latex particles. In some embodiments, the detectable label can comprise a small molecule such as biotin.

The methods of the present invention comprise detecting the presence of the one or more binding agents on the solid substrate. The presence of the one or more binding agents indicates the presence of the target molecule in the liquid sample. In some embodiments, detecting the presence of the one of more binding agents comprises detecting the presence of the detectable label.

A signal from the detectable label can be detected using any suitable method known in the art. Exemplary methods can include, but are not limited to, visual detection, fluorescence detection (e.g., fluorescence microscopy), scintillation counting, surface plasmon resonance, ellipsometry, atomic force microscopy, surface acoustic wave device detection, autoradiography, and chemiluminescence. In a specific embodiment, the detection can be done using a Genepix 4300A (Molecular Devices, San Jose, CA). The methods of detection may depend upon the nature of the detectable label, if one is used in the methods of the present invention.

In some embodiments, the methods of the present invention further comprise quantifying the amount of the detectable label to provide a measure of the target molecule in the liquid sample. Quantification can be done using methods and techniques known in the art. Quantification of the signal can be relative or absolute. In absolute quantification, no reference sample or comparison to other samples are needed. The target molecule can be directly quantified with precision determined by any suitable method known in the art, such as the ones provided above. An absolute quantification can also use the standard curve method. In this method, one quantitates unknowns based on a known quantity. For example, a standard curve is created using samples with known concentrations of a target molecule. Then, one compares a test sample to the standard curve and extrapolates a value. In relative quantification, one analyzes changes in a target molecule in a given sample relative to another sample, such as a reference sample. A reference sample as used herein can be, for example, a sample with known concentration of a target molecule, a sample from a different subject, or a sample from the same subj ect.

The measurement of the target molecule may be expressed as a qualitative value, or more likely as a quantitative value. As used herein, the quantification of the target molecule can be a relative or absolute quantity. Of course, the quantity (concentration) of any of the target molecule may be equal to zero, indicating the absence of the particular target molecule sought. The quantity may simply be the measured signal, e.g., fluorescence, without any additional measurements or manipulations. Alternatively, the quantity may be expressed as a difference, percentage or ratio of the measured value of the particular target molecule to a measured value of another compound including, but not limited to, a standard or another target molecule. The difference may be negative, indicating a decrease in the amount of measured target molecule(s). The quantities may also be expressed as a difference or ratio of the target molecule(s) to itself, measured at a different point in time. The quantities of target molecule may be determined directly from a generated signal, or the generated signal may be used in an algorithm, with the algorithm designed to correlate the value of the generated signals to the quantity of target molecule(s) in the sample.

The methods of present invention do not require a separate sample extraction step after the sample is dried on a solid substrate. For example, dried liquid biological samples, such as dried blood or serum spots on filter paper or glass slides, have been used in the field. However, when followed with a bioassay, these drying liquid sample approaches all require or contemplate a separate sample extraction step and the sample is not dried on the pre-spotted detection array or device itself.

### EXAMPLES

### Example 1: Ferritin Assay

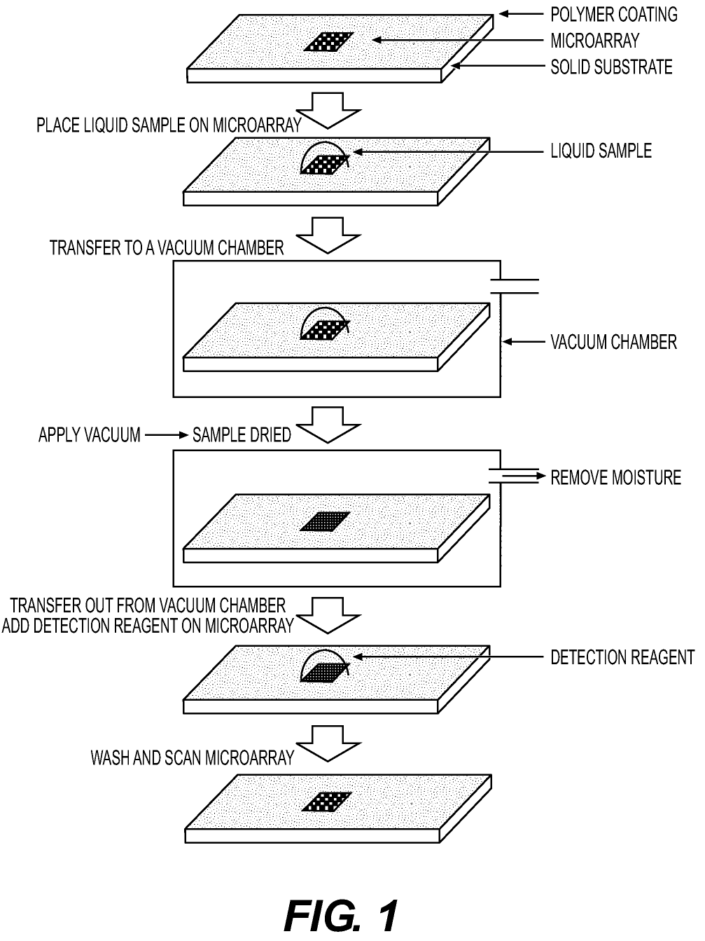

FIG. 1 shows an exemplary SCA process. Briefly, a sample, e.g., a liquid sample, is added to the solid substrate, such as a microarray surface, and dried quickly with assistance of vacuum. The drying process forces the target molecules from the liquid sample solution towards the microarray surface to facilitate capture. After the sample is dried on the array surface, binding agents labeled with a detectable label are added to the surface to label the captured target molecules. The remaining components from the assay can be washed away later to result in a clean surface with the labeled target molecules.

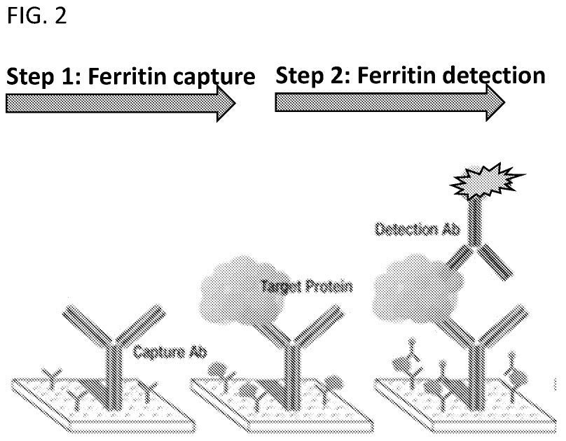

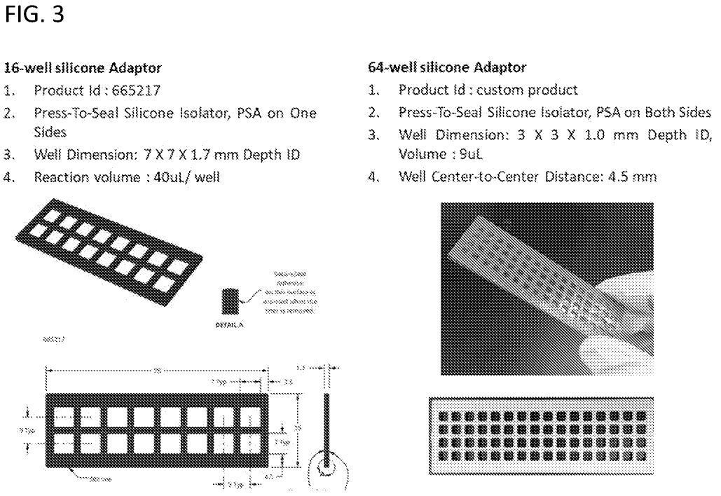

To demonstrate the applicability of the SCA method, it was applied to Ferritin quantitative sandwich assay as shown in FIG. 2. In this assay, 6x6 array of Ferritin capture antibody was spotted on a 25x75.6 mm glass slide coated with e-POEGMA (US6096319A). A total of 64 6x6 arrays were spotted on each slide. As show in FIG. 3, the arrays were separated with silicon adaptor, with adhesive to adhere to the slide surface, to create 64 individual wells. Each well enclosed a 6x6 array. In this setting, a maximum of 64 samples can be tested on a single slide. The maximum volume for each well is 9 microliters (µL).

The Ferritin microarray printing procedure is described briefly herein.
(1) Capture mIgG at 0.5 mg/mL was printed as 6x6 array with spot-to-spot distance of 400 um.
(2) 64 arrays were printed on a Nexterion slide coated with e-POEGMA. The center-to-center distance between arrays is 4500 um.
(3) Humidity in Printer was about 50-55%.
(4) Post Printing Wait: Slides on printer stage for 1 hour.
(5) Post Printing Incubation: Overnight at 40° C.
(6) Storage: vacuum sealed with desiccant, 2-8° C.

Further, an exemplary Ferritin assay procedure is provided below:
(1) Sample preparation:
   a. Test human serum at 1 to 2 dilution with ThermoScientific diluent (TSB).
   b. Dilute Recombinant Ferritin (Meridian) in TSB contains 0.05% Tween20.
(2) Assemble the GraceBio 64-well silicone adaptor (with adhesive) on the slide with printed array.
(3) Pre-wash ePOEGMA slide using in PBS-T 0.1%.
(4) Add µL of sample in each well on the slide. The comparison wet assay uses 7 µL of the same samples added to the array and incubated for 30 min at 37° C., on orbital shaker (300 rpm), without drying.
(5) Dry the samples on the arrays with vacuum for 8 min at 37° C.
(6) Add 7 µL AF647 labeled Anti-hFerritin IgG (3, 6, 9 µg/mL, mAb5), incubate for 10 minutes at 37° C. on orbital shaker (300 rpm).
(7) Wash manually 3 times with PBS (contains 0.1% tween-20) using multi-channel pipettor and vacuum manifold.
(8) Remove the silicone adaptor and perform final rinse with PSB in a 50 mL conical tubes.
(9) Dry the slides by centrifugation (1000 g, 30 seconds) in 50 mL conical tubes.
(10) Scan on the Genepix 4300A (Molecular Devices, San Jose, CA) at 635 nm.

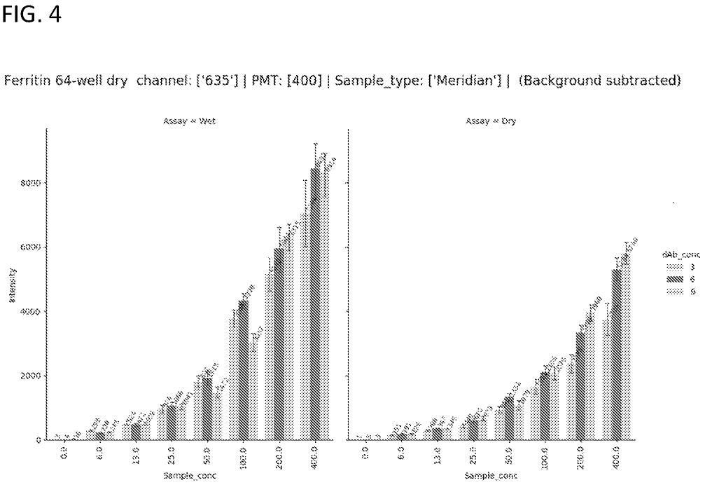

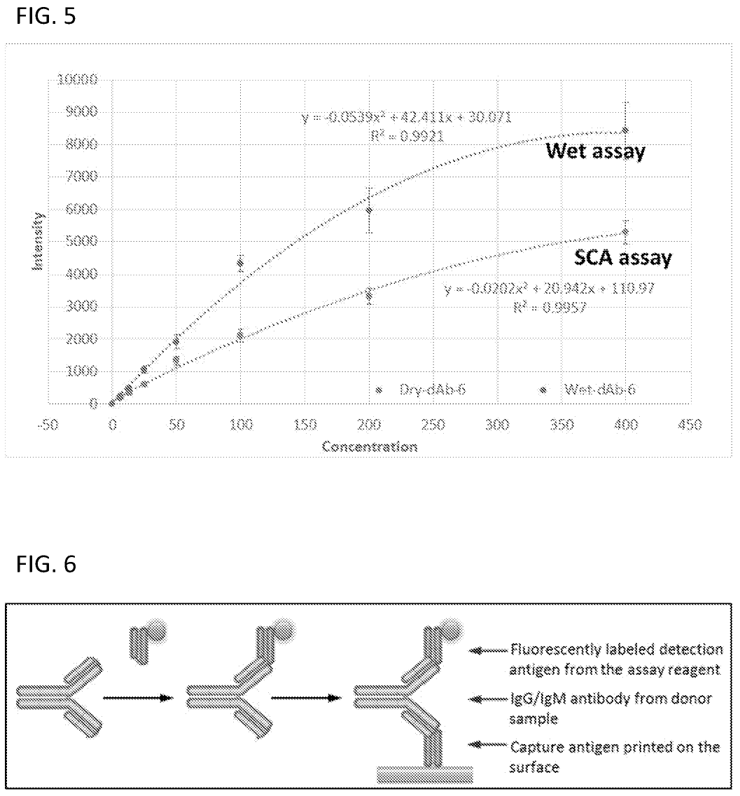

FIG. 4 shows the assay intensity from a recombinant Ferritin (Meridian Life Sciences, Memphis, TN) at a series of concentrations, 0-400 ng/mL. The SCA assay was performed side-by-side with typical wet assay on identical slides. The assay intensity forms the same trend for both SCA and wet assay. The scatter plots in FIG. 5 shows that the SCA and wet assay intensities can be fit with 2^{nd} order polynomial and the SCA assay intensity is lower, but the assay variation (the error bar) is also smaller.

### Example 2: Double-Antigen Bridging Immunoassay

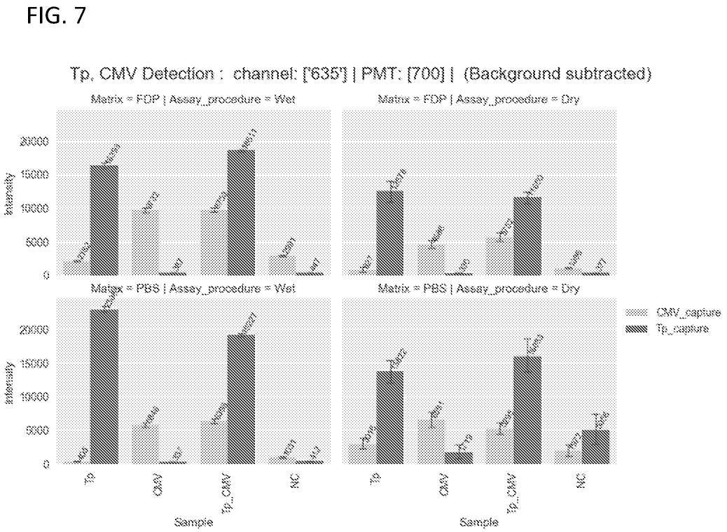

The SCA method was also applied to double-antigen bridging immunoassay. As shown in FIG. 6, in double-antigen bridging immunoassay antigen was spotted as array to capture the specific antibody targets from the sample. Labeled antigen was used to detect the captured antibody targets.

The instant example was a duplex detection assay which simultaneously detected Treponema pallidum (Tp) and Cytomegalovirus (CMV) specific antibodies from human serum or plasma samples.

In this assay, antigens specific to Tp and CMV antibodies were spotted in alternate columns in a 10×10 array on e-POEGMA coated glass slides. Sixteen such arrays were spotted on each slide coated with e-POEGMA. As show in FIG. 3, the arrays were separated with silicon adaptor (with adhesive to adhere to the slide surface) to create 16 individual wells. Each well encloses a 10×10 array. In this setting, a maximum of 16 samples can be tested on a single slide. The maximum volume for each well is 80 µL. The assay procedure is briefly described herein:
(1) Array printing
   i. Recombinant Tp antigen (Meridian, R01705-4) and CMV antigen (Rekom, RAG0110) at 0.5 mg/mL in PBS with 0.01% Tween-20) were spotted and immobilized in alternate columns in a 10×10 array on e-POEGMA coated glass slides, 50 spots for each antigen.
   ii. 16 arrays were printed on a Nexterion slide coated with e-POEGMA. The center-to-center distance between arrays was 9000 µm.
   iii. The other printing conditions were identical to that for Ferritin as provided in Example above.
(2) Simulated samples were constituted by spiking rabbit anti-Tp polyclonal IgG antibodies J120, and/or mouse anti-CMV-pp28 monoclonal IgG2a (kappa), mouse anti-CMV-pp65 monoclonal IgG2a (kappa) antibody at the final concentration of 50 ug/mL and 5 ug/mL, respectively, in Ferritin depleted human plasma. All three antibodies are obtained from East Coast Bio (North Berwick, ME).
(3) Assemble the GraceBio 16-well silicone adaptor (with adhesive) on the slide with printed array.
(4) Pre-wash the wells manually with 1x PBST for 3 times. Dry the slide by centrifugation.
(5) Mix the samples with dilution buffer (PBST with 1% BSA) containing AF647-labeled Tp-dAg and CMV-dAg (0.25 ug/mL).
(6) Add 4 µL sample per well; dry on the arrays with vacuum for 10 min at room temperature.
(7) Wash manually 3 times with PBS (contains 0.1% tween-20).
(8) Remove the silicone adaptor and perform final rinse with PSB in a 50 mL conical tubes.
(9) Dry the slides by centrifugation (1000 g, 30 seconds) in 50 mL conical tubes.
(10) Scan on the GenePix 4300A at 635 nm.

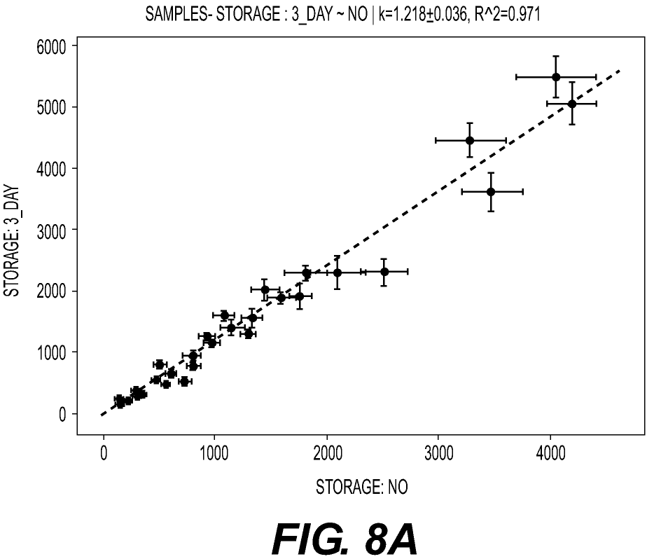

FIG. 7 shows the assay intensities from Tp and CMV specific capture antigen spots from 4 different samples, solution spiked with Tp antibody only, spiked with CMV antibody only, spiked with both Tp and CMV antibodies, and solution without any (negative control, NC). The assay is performed in dry (SCA) and wet format side-by-side. The wet assay uses 40 µL of samples and the SCA assay uses 4 µL samples.

For each sample in each assay format, two matrices are compared, sample with PBS buffer only and sample with 30% Ferritin depleted plasma (FDP) and PBS buffer. The result shows that for the samples with FDP matrix the SCA assay is comparable with the wet assay, both of which showed specific detection of Tp and CMV antibodies with reasonably low background. For the SCA assay without FDP matrix, there is nonspecific signal for both Tp and CMV.

### Example 3: Storage of Dried Samples on Array Substrate

This example demonstrates that, during the SCA sandwich assay, samples dried on the array substrate may be stable for extended time, which allows flexible waiting time between the capture step and the detection step shown in FIG. 2. The detection does not have to follow the capture step immediately. An indefinite waiting time between the 2 steps can be applied, thus make the assay process flexible. For example, samples collected on the array substrate can be stored overnight or for longer time before detection. For example, as shown here, it is feasible to store the samples dried on the array substrate for 3 days before detection.

Following the same procedure as described in Example 1 above, a set of 48 human plasma/serum samples along with a reference recombinant Ferritin standards from world health organization (WHO) at a series of concentration from 0 to 200 ng/mL were prepared and tested on 2 slides. The tests on the 2 slides were identical with the following exception. For one slide, the assay was completed without delay between the detection and capture steps. For the other slide, after sample drying, it was stored at 4° C. for 3 days before detection.

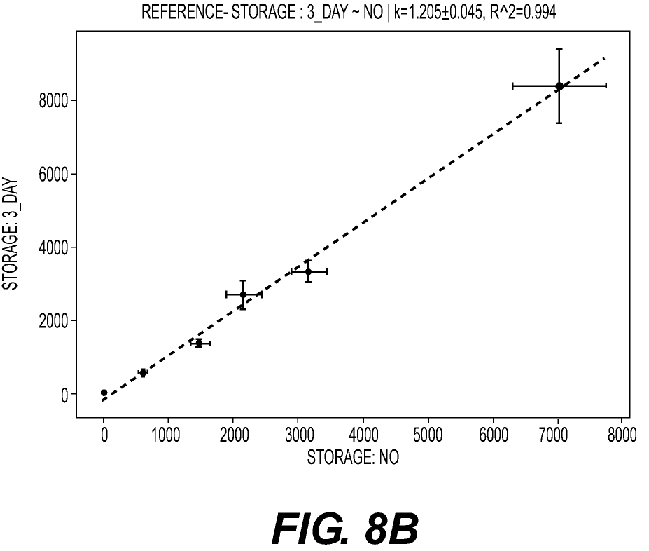

The assay intensities from the two slides are plotted in FIGS. 8A (plasma/serum) and 8B (WHO standard reference). The intensity from the slides with 3 days storage was highly correlated to that from the slide with immediate detection. It was observed that the intensity for both the standard reference and the plasma/serum samples were about 20% higher after being stored for 3 days. It should be noted that the absolute assay intensity may have slide-to-slide variation. Practically, the intensity from the samples can be scaled with the reference standard intensity to reduce the slide-to-slide variation.

The results showed that the Ferritin samples dried on the array substrate is stable for detection after 3 days storage at 4° C.

### Example 4. Human Interleukin 6 Quantitative Assay

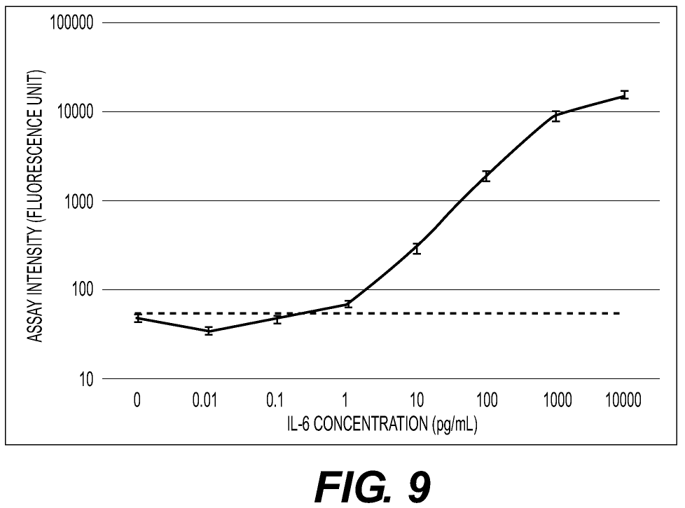

In this example, the SCA method has been applied in an assay to determine human Interleukin 6 (IL-6) recombinant antigen. In this application, the anti-IL6 monoclonal antibody was spotted and immobilized in a 10x10 array format on e-POEGMA coated glass slides. The samples were prepared by diluting the recombinant IL-6 antigen to the final concentration of 0.01, 0.1, 1, 10, 100, and 1000 pico-gram/mL, respectively, in Phosphate Buffered Saline (PBS) with 10% fetal bovine serum. To detect the IL-6 content, 4 µL of the sample solution was pipetted on the IL-6 antibody microarray well. The slide was then placed in a desiccator to dry under vacuum for 10 minutes at room temperature. After the samples were dried, 40 µL of biotinylated IL-6-specific monoclonal antibody at 5 microgram/mL concentration was applied to each array and incubated for 60 minutes at room temperature. After incubation, the reaction mixture was aspirated, and 40 µL Alexa Fluor 647 conjugated streptavidin was added to the microarray and incubated for 30 minutes. The slide was subsequently rinsed with PBS containing 0.1% Tween-20 for 3 times followed by final rinse with PBS, and dried by brief centrifugation. Fluorescent signal from the microarrays was then determined by using the GenePix fluorescent scanner at 635 nm wavelength. The average fluorescence intensity from the array spots for each sample test was plotted in FIG. 9. The graph indicates a detection limit of 1 pico-gram/mL.

### Example 5. ABO Antibody Detection Assay

In this example, the SCA method has been applied in identification of the A and B-blood type antibodies in human plasma, which is also known as the reverse ABO blood typing.

In this application, biotinylated synthetic Type A or Type B antigen was mixed with streptavidin in 4:1 ratio. The streptavidin-A/B complexes were spotted on e-POEGMA coated glass slides in the same microarray. The microarray slide was blocked with the blocking buffer containing phosphate buffered saline (PBS) with 1% BSA and 0.05% Tween-20 by drying in vacuum for 25 minutes at 37° C.

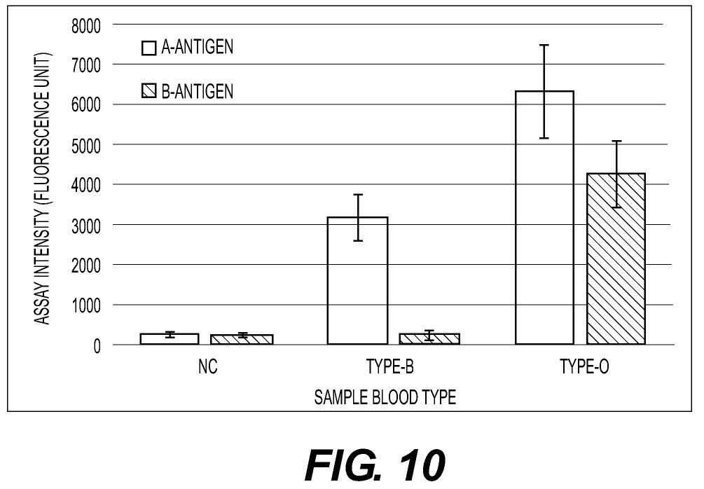

Plasma samples from donors with Type B and Type-O blood types were tested by first diluting the samples 1:3 in the dilution buffer. 4 µL of the diluted samples were pipetted on individual microarrays of the A or B synthetic antigen on the slide. The slide was then placed in a desiccator to dry under vacuum for 10 minutes at room temperature. After the samples were dried, 40 µL of Alexa Fluor 647 conjugated Goat-anti-Human IgM was applied to each microarray and incubated for 10 minutes at room temperature. The slide was subsequently rinsed by using PBS containing 0.1% Tween-20 for 3 times followed by final rinse with PBS, and dried by brief centrifugation. Fluorescent signal from the microarrays was then determined by using the GenePix fluorescent scanner at 635 nm wavelength. The fluorescence intensity from the array spots with streptavdin/A-antigen and B-antigen for each plasma sample was plotted in FIG. 10 below. The A-antigen microarray spot intensity indicates presence of anti-A antibody from the donor with Type-B blood; the B-antigen microarray spot intensity indicates the presence of anti-B antibody from the donor with Type-A blood. As shown in FIG. 10, as expected, the Type-B donor plasma sample resulted A-antigen intensity but no B-antigen intensity, indicating the presence of anti-A antibody. On the other hand, the Type-O donor plasma showed fluorescence assay intensity for both A and B antigens, indicating presence of both anti-A and anti-B, which is consistent with the Type-O blood type.

1. A method for detecting a target molecule in a liquid sample, the method comprising:
(a) applying at least a portion of the liquid sample on a solid substrate that comprises a non-fouling polymer layer;
(b) decreasing the atmospheric pressure surrounding the solid substrate containing the portion of the liquid sample for a time sufficient for a majority of the liquid to evaporate from the portion of the liquid sample applied to the solid substrate;
(c) contacting the liquid sample with one or more binding agents that specifically binds to the target molecule after the majority of the liquid has evaporated from the liquid sample; and
(d) detecting the presence of the one or more binding agents on the solid substrate; wherein the presence of the one or more binding agents indicates the presence of the target molecule in the liquid sample.

2. (canceled)

3. The method of claim 1 or 13, further comprising transferring the solid substrate into a sealable enclosure that is connected with a vacuum after placing the portion of the liquid sample on the solid substrate.

4. The method of claim 3, wherein the sealable enclosure is a desiccator.

5. The method of claim 4, wherein the temperature is from about 4° C. to about 40° C. when the atmospheric pressure is decreased.

6. The method of claim 5, wherein the atmospheric pressure is reduced to from about 0 to about 1000 millibar.

7. The method of claim 6, wherein the time sufficient for a majority of the liquid to evaporate from the portion of the liquid sample applied to the substrate is less than about 10 minutes.

8. The method of claim 7, wherein the time sufficient for a majority of the liquid to evaporate from the portion of the liquid sample applied to the substrate is less than about 5 minutes.

9. The method of claim 8, wherein the liquid sample is a biological sample selected from the group consisting of: blood, serum, plasma, lymph fluid, bile fluid, urine, saliva, mucus, sputum, tears, cerebrospinal fluid (CSF), bronchioalveolar lavage, nasopharyngeal lavage, rectal lavage, vaginal lavage, colonic lavage, nasal lavage, throat lavage, synovial fluid, semen, ascites fluid, pus, maternal milk, ear fluid, sweat, and amniotic fluid.

10. The method of claim 9, wherein the solid substrate is selected from the group consisting of glass, silicon, a metal, a plastic, and a polymer.

11. The method of claim 10, wherein the non-fouling polymer layer comprises a co-polymer of epoxy-co-poly(oligo(ethylene glycol) methyl ether methacrylate (e-POEGMA).

12. The method of claim 1, wherein the non-fouling polymer layer further comprises a plurality of capture regions, wherein each of the plurality of capture region comprises at least one distinct population of capture agents, and wherein the at least one distinct population of capture agents specifically binds to the target molecule in the liquid sample.

13. The method of claim 12, wherein each of the plurality of capture regions comprises more than one distinct population of capture agents.

14. The method of claim 10, wherein the capture agent is a cell, a small molecule ligand, a lipid, a carbohydrate, a polynucleotide, a peptide, a protein, an antigen, an antibody, or a combination thereof.

15. The method of claim 14, wherein the target molecule is a cell, a small molecule ligand, a lipid, a carbohydrate, a polynucleotide, a peptide, a protein, an antigen, an antibody, or a combination thereof.

16. The method of claim 15, wherein the target molecule is a blood type antigen, a platelet antigen, an infectious disease antigen, a human leukocyte antigen (HLA), an interleukin antigen, or any combination thereof.

17. The method of claim 16, wherein the target molecule is a human immune deficiency virus (HIV) antigen, a hepatitis B virus (HBV) antigen, a hepatitis C virus (HCV) antigen, a human T-lymphotropic virus (HTLV) antigen, a Treponema pallidum (TP) antigen, or any combination thereof.

18. The method of claim 16, wherein the target molecule is human A blood type antigen, a human B blood type antigen, a human AB blood type antigen, a human O blood type antigen, a human Rh factor antigen, a human MNS blood type antigen, a human P blood type antigen, a human P1PK blood type antigen, a human Lutheran blood type antigen, a human Kell blood type antigen, a human Lewis blood type antigen, a human Duffy blood type antigen, a human Kidd blood type antigen, a human Diego blood type antigen, a human Yt or Cartwright blood type antigen, a human Xg blood type antigen, a human Scianna blood type antigen, a human Dombrock blood type antigen, a human Colton blood type antigen, a human Landsteiner-Wiener blood type antigen, a human Chido/Rodgers blood type antigen, a human H blood type antigen, a human Hh/Bombay blood type antigen, a human Kx blood type antigen, a human Gerbich blood type antigen, a human Cromer blood type antigen, a human Knops blood type antigen, a human Indian blood type antigen, a human Ok blood type antigen, a human Raph blood type antigen, a human John Milton Hagen blood type antigen, a human I blood type antigen, a human li blood type antigen, a human Globoside blood type antigen, a human Gill blood type antigen, a human Rh-associated glycoprotein blood type antigen, a human Forssman blood type antigen, a human Langereis blood type antigen, a human Junior blood type antigen, or any combination thereof.

19. The method of claim 16, wherein the one or more binding agents are labeled with a detectable label.

20. The method of claim 19, wherein the detectable label is a chromophore, a fluorophore, a biotin, a radiolabel, a polynucleotide, a small molecule, an enzyme, a nanoparticle, a microparticle, a quantum dot, or an upconverter.

21. The method of claim 20, wherein detecting the presence of the one of more binding agents comprises detecting the presence of the detectable label.

22. The method of claim 21, further comprising quantifying the amount of the detectable label to provide a measure of the target molecule in the liquid sample.

23. The method of claim 22, further comprising storing the sample on the solid substrate for a period of time after the majority of the liquid has evaporated from the portion of the liquid sample applied to the solid substrate and before contacting the liquid sample with one or more binding agents.

24-26. (canceled)
